(19) United States
(12) Patent Application Publication
(10) Pub. No.: US 2012/0179096 A1
(43) Pub. Date: Jul. 12, 2012

(54) Locking Syringe System

(75) Inventor: Andreas Aeschlimann, Caulfield North (AU)

(73) Assignee: SSB TECHNOLOGY PTY LTD, Perth, Western Australia (AU)

(21) Appl. No.: 13/498,120

(22) PCT Filed: Sep. 24, 2010
(86) PCT No.: PCT/AU2010/001257

§ 371 Date: Mar. 23, 2012

(30) Foreign Application Priority Data
Sep. 25, 2009 (AU) 2009904666

(51) Int. Cl. A61M 5/31

(52) U.S. Cl. 604/87

(57) ABSTRACT

The invention provides methods and apparatus for reconstituting fluids and substances and injecting the reconstituted materials. Apparatus may include an injection needle having a hub, a draw-up needle having a hub, and a safety cap for engagement with a syringe barrel or reservoir for transfer and/or reconstitution and injection of fluids. The apparatus includes engagement means for securely engaging the hubs of the needles and/or cap to minimise inadvertent pricks with either of the needles during the filling of a syringe with reconstituted materials and injecting the materials. Preferably the engagement means incorporates a channel on one hub and a protrusion on the other hub. The system may incorporate a second bore for quick draw-up of materials and fluids for injection. Preferably, the apparatus comprises of polymeric materials which can be injection moulded. The invention includes methods for drawing-up and reconstituting fluids for injection.

### FIELD OF INVENTION

This invention relates to the field of cannulae, and needles and syringes for transferring fluids.

### PRIORITY CLAIM

This document claims priority from Australian provisional application no. 2009904666 filed on 25 Sep. 2009, the contents of which are herein incorporated by reference.

### BACKGROUND TO THE INVENTION

Drawing up fluids for safe injection into a subject has long been of interest in the medical profession and millions of syringes are used and disposed of each year. There are a number of problems with currently available syringes. The narrow gauge needle that is necessary to effectively penetrate a subject for delivery of a fluid with minimal pain often causes injuries to professionals administering the fluids. The problem is exacerbated for reconstitution processes where the needle must first penetrate a septum of a fluid-containing vial and the several steps necessary to penetrate the vial to draw up fluid for combining with another fluid or dry substance for injection to a subject provide many opportunities for needle stick injuries to occur. Prior art needles often suffer the problem of coring of a septum when drawing up fluid for injection or even coring of the subject's skin. Further, needle stick injuries can cause inadvertent infection or cross-contamination of previously sterile fluids in the draw-up or injection activities.

The prior art includes a number of solutions to the needle-stick problem and resulting cross-contamination or infection. For example, there are retractable needles available for the problem. One problem with such retractable needles is that they are often single use and complex. The complexity of the operation of such needles makes manufacturing processes complex and therefore expensive, resulting in expensive syringes.

Reconstitution syringes present extra problems with extra strength needed for penetration and withdrawal of syringes through sealing septa, for example, to fill a syringe before injection. WO 2008/057361 describes a solution to this problem, being the addition of force bearing wings or finger flanges and lugs on the needle or cannula to facilitate the process and transfer the extra force needed for this process.

It is known in the art to use two hypodermic needles for the draw-up and injection in reconstitution and injection processes. The first needle, usually a bigger gauge needle, is used to draw-up from the vial. The draw-up needle is then exchanged with a smaller gauge needle which is used for injection.

Hypodermic needles are lubricated with silicone lubrication, which may create problems. The lubrication is meant to decrease the pain during injection. However, some of the silicone lubrication may be pushed off when the needle is used to penetrate the vial stopper (septum) and the needle which is used to draw-up may exhibit a reduced lubrication affect.

What is needed are improved methods for reconstituting materials for injection, and apparatus or systems for making it easier and convenient to reconstitute materials for injection, and injecting materials.

### BRIEF DESCRIPTION OF THE DRAWINGS

FIG. 1 shows an exploded view of an embodiment of the invention.

FIG. 2 shows a longitudinal cross-section of a locking syringe system.

FIG. 3 shows a perspective view of an injection needle and hub.

FIG. 4a shows a perspective view of a syringe barrel having a Luer lock distal portion.

FIG. 4b shows a close-up perspective view of a Luer lock engagement portion of a syringe barrel.

FIG. 5a shows a perspective view of the outer surface of an embodiment of a draw-up needle.

FIG. 5b shows a perspective cut-out portion and view of the inner surface of the draw-up needle in FIG. 5a.

FIG. 6 shows a top perspective view of a locking syringe system kit.

FIG. 7 shows a second embodiment of the invention in exploded view.

FIG. 8 shows the second embodiment of the invention in engaged perspective view.

FIG. 9a shows a perspective view of a second embodiment of a draw-up needle.

FIG. 9b shows a perspective view of the second embodiment of a draw-up needle with a portion cut away.

FIG. 10 shows a perspective view of second embodiment of an injection needle and hub.

FIG. 11a shows a longitudinal cross-section of the second embodiment of a locking syringe system in engaged position.

FIG. 11b shows a longitudinal cross-section of the second embodiment of a locking syringe system in disengaged position.

FIGS. 12a and 12b shows in perspective view the second embodiment of a locking syringe system in engaged position with a portion cut away.

FIG. 13 shows a cap for a syringe system.

FIGS. 14a to 14d show steps in the operation of an embodiment of a locking syringe system.

### SUMMARY OF THE INVENTION

The invention provides a syringe system which advantageously enables fluids to be drawn through one or two needles into a reservoir and be ready for injection into a subject with a limited number of steps. In particular, the syringe system may comprise of a draw-up needle and an injection needle engaged with the reservoir. The system may include a cap to cover the needles and prevent unintentional injury or cross-contamination of the injection needle. Most advantageously, the invention may provide a second entry channel into the reservoir for enhanced draw-up of fluid into the reservoir in some embodiments. It is an object of the invention to provide a syringe system that reduces the possibility of unintended needle pricks during draw-up of fluids for injection or reconstituting materials for injection. It is a further object of the invention to provide an improved method of drawing up fluids from vials for injection. It is a further object of the invention to provide simple and methods for reconstituting materials for injection.

In one aspect, the invention provides syringe for transferring fluid comprising: at least one cannula incorporating a barrel and hub wherein the syringe incorporates at least two channels for fluid transmission.

In another aspect, the invention provides apparatus for transferring fluid comprising of a first needle for drawing-up fluid; an injection needle; wherein the injection needle comprises of a cannula and a tip having an aperture disposed opposite a blade.

In another aspect, the invention provides apparatus for transferring fluid comprising of a first cannula including a hub; a second cannula including a second hub; wherein said hubs of said cannulae are engaged with engagement means. Preferably the engagement means comprises of a protrusion of said first hub engaging an engagement surface of said second hub. Preferably apparatus further comprises of a channel in said second hub for axially guiding said protrusion to a rotating position. Preferably, the apparatus further comprises of an engagement surface on said second hub wherein the engagement is effect by rotating said first hub into engagement position. Preferably, the apparatus further comprises of a liquid reservoir such as a barrel of a syringe. Preferably, the apparatus further comprises of a engagement means for engaging a reservoir with the first and second cannula. Preferably, the engagement means for engaging the reservoir with the cannulae is a Luer lock. Most preferably, the apparatus incorporates a cap. The apparatus comprise entirely of polymeric materials that are suitable for injection moulding. However, the needles may alternatively comprise of metallic materials.

The invention further may comprise of external packaging incorporating the various combinations of elements.

In a still further aspect, the invention provides a method for mixing substances comprising the steps of drawing a fluid through at least two channels simultaneously and mixing the fluid with another substance. The method may further comprise the step of injecting the mixture into a subject. The method may be used with at least one substance that is pharmaceutically active.

In a still further aspect, the invention provides a method of engaging two needles having hubs one within the other, the method including the steps of: axially moving the hub of a first needle into engaging position on the hub of a second needle; and rotating the hub of the second needle into secure engagement. The method is most advantageously used when the needles comprise of a draw-up needle and an injection needle.

All parts of the syringe system may be manufactured from polymeric materials using the method disclosed in U.S. Pat. No. 5,620,639, for example, or alternative methods using polymer-injection techniques. When the parts are polymeric material, they can be easily disposed of for recycling of the materials, if desired. Alternatively, the needle may be made of metal. Preferably the metal is steel. Including metal parts in the system, however, reduces the potential to recycle used syringe materials because of the co-mingling of the different materials.

### DETAILED DESCRIPTION OF THE PREFERRED EMBODIMENTS AND FIGURES

The objects of the invention are best understood with reference to the embodiments described herein and with reference to the figures. It will be understood by those skilled in the art that the invention is not limited to the embodiments shown in the figures but includes embodiments not illustrated but within the scope of the claims appended hereto.

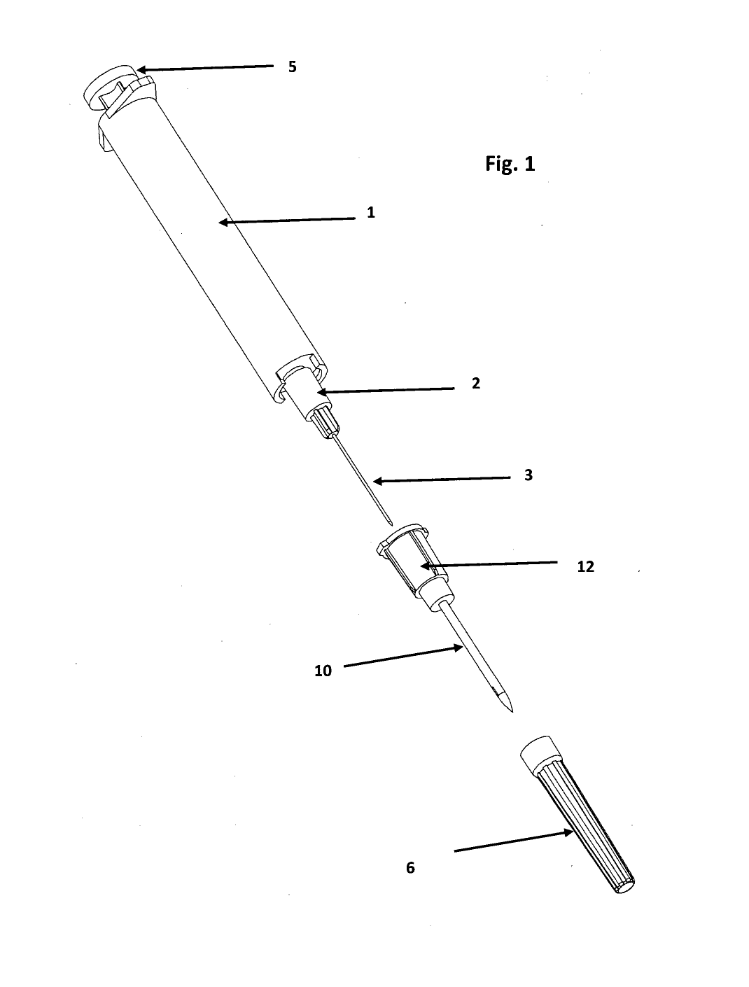

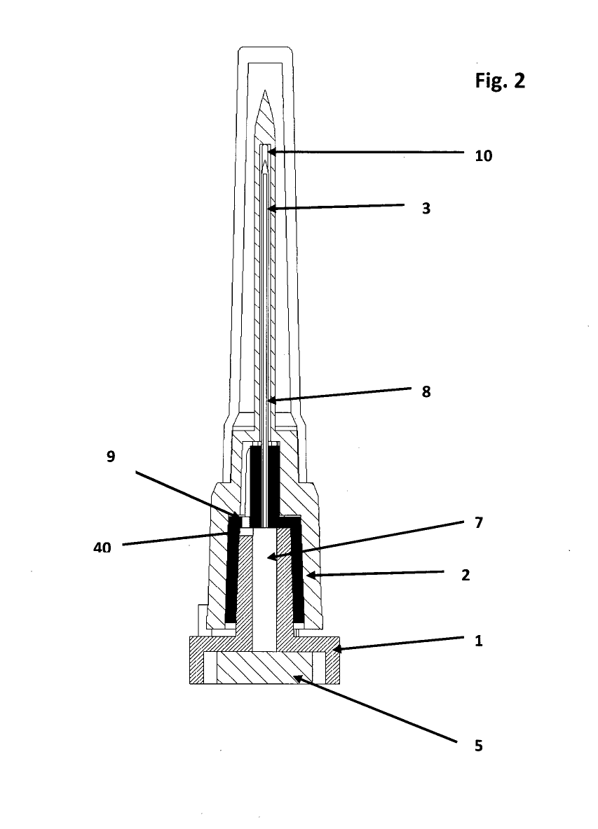

FIG. 1 shows an exploded view of an embodiment comprising the main elements of the invention, comprising of a reservoir or syringe barrel 1, injection needle hub 2, injection needle 3, draw-up needle 10, and piston 5. An embodiment may include a cap 6. As shown in FIG. 2 in transverse longitudinal section of a preferred embodiment of the invention, the syringe barrel or reservoir 1 includes a first channel 7 which forms a conduit with the channel of the injection needle 8. The syringe barrel may incorporate at least a second channel or bore 9, the aperture of the second channel or bore 9 being sealable as herein described. The bore 9 is in fluid communication with a further channel 40 which also forms a conduit with the first channel 7 to the reservoir 1 of the syringe. The bore 9 provides a convenient conduit for drawing-up fluid through a channel 40 in addition to the channel 8 provided by the injection needle 3. In the most preferred embodiment incorporating the second channel 40 in fluid communication with the first channel 7, the cooperation of the bore 9 and second channel 40 into the first channel 7 and injection needle channel 8 may most advantageously significantly increase the volume of fluid drawn into the reservoir with a single draw-up pass of the piston. It will be understood that of embodiments may include further bores 9 and channels 40 in similar fluid communication with the reservoir 1 through the first channel 7. Most advantageously the reduced effort needle to complete a draw-up pass to fill the reservoir helps to increase the efficiency of filling the reservoir.

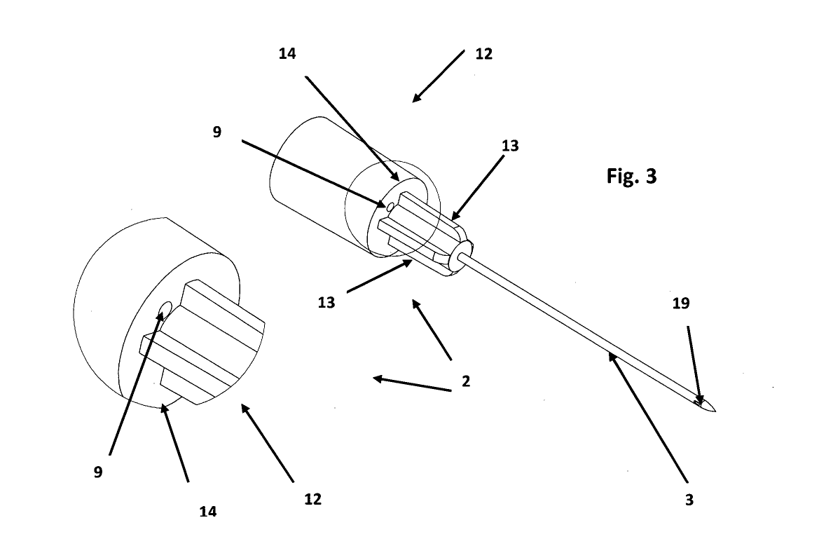

An embodiment of the bore is shown in FIG. 3 where it is located on the hub of the injection needle. The invention includes one or more such apertures or bores to define channels for passage of fluid between the reservoir and the needle. For example, there may be two or more apertures. In operation, with the draw-up needle 10 engaged with the injection needle hub 2 the draw-up needle 10 is oriented so that the entry to the bore is open and the bore provides a channel for fluid communication between the draw-up needle and the reservoir so that fluid is drawn into the reservoir in extra volume. The extra volume can be taken up through the extra channels defined by the at least one bore into the reservoir will be defined by the total diameter of the bore. The bore is then closed as described herein and the fluid in the reservoir can be injected into a subject through the injection needle.

Most advantageously, the invention provides a method of transferring fluid from a container to a subject comprising of the step of drawing fluid into a syringe barrel or reservoir through multiple channels in a single draw-up pass and the step of administering the fluid to a subject by a single injection pass. Preferably, the draw-up step includes drawing fluid concurrently through two channels into a reservoir. Preferably, the administration step includes administering fluid to a subject through a single channel. The invention may provide a method of filling a reservoir with fluid and administering the fluid to a subject comprising of the step of engaging a plurality of needles defining parallel channels to a reservoir, the step of drawing fluid into the reservoir through the needles simultaneously, the step of disengaging a first needle from the reservoir and the step of administering the fluid from the reservoir to a subject with a second needle.

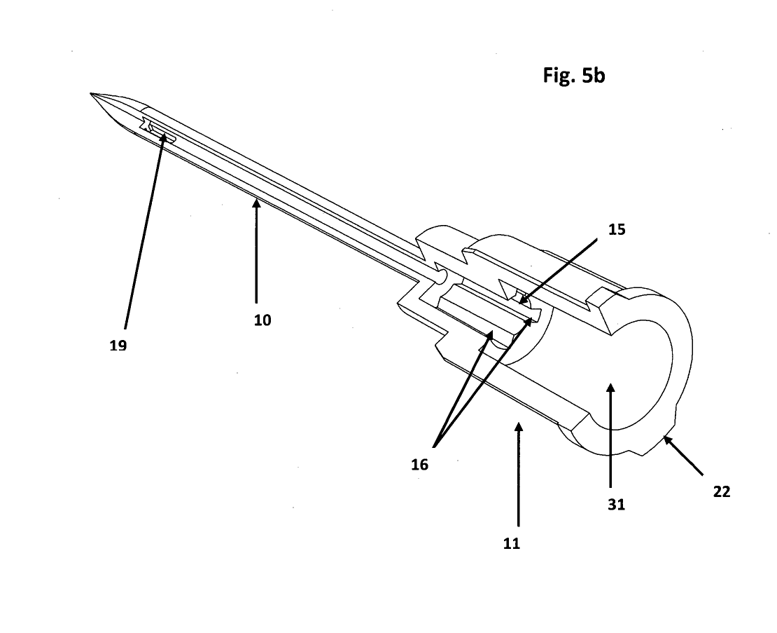

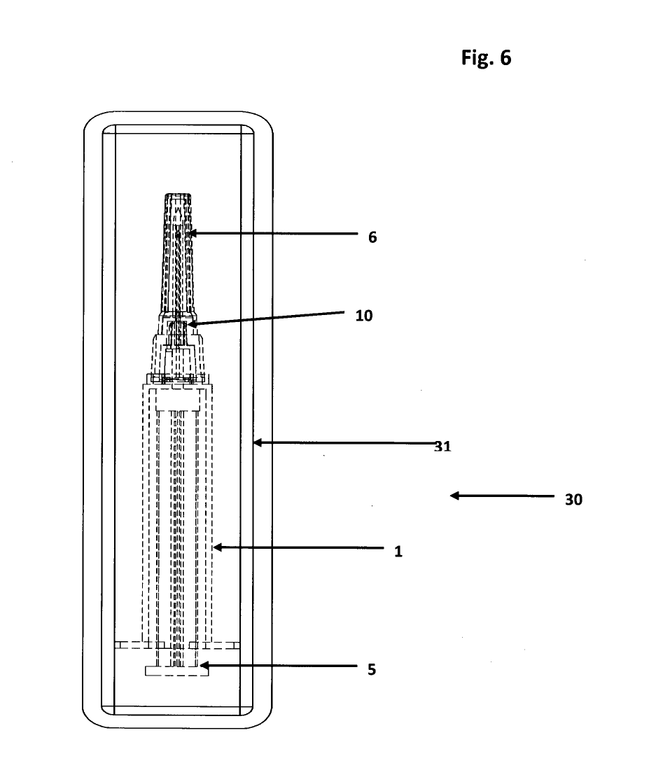

As shown in FIG. 5a, the draw-up needle 10 comprises of a hub 11 and a needle or cannula 4. Preferably, the hub comprises of a distal portion 17 with a reduced diameter compared to a proximal portion 18. The reduced diameter of the distal portion 17 allows engagement of a cap 6 over the draw-up needle 4. The cap, if present, may prevent unintended needle-stick injuries and cross-contamination which may occur with an uncovered draw-up needle. The hub 11 may incorporate protrusions 21 on its external surface, the protrusions being be helpful for a user to better grip the draw-up needle in use. Preferably, the protrusions 21 are rib-shaped, but may take other shapes, such as nubs. As shown in FIG. 5b, the inner surface of the draw-up needle hub 11 incorporates intrusions 16 for complementarily engaging the outer surface 12 (shown as four protrusions) of the hub of the injection needle.

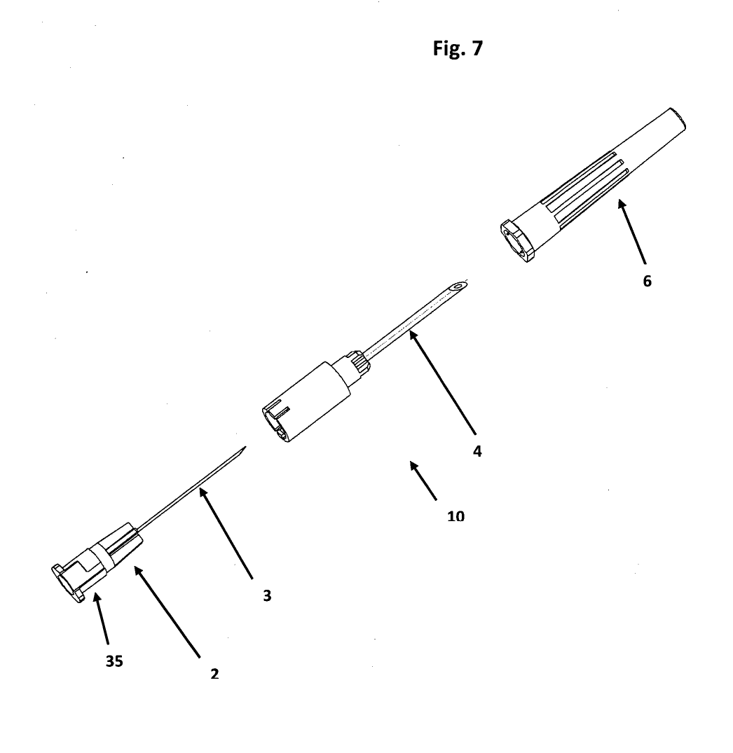

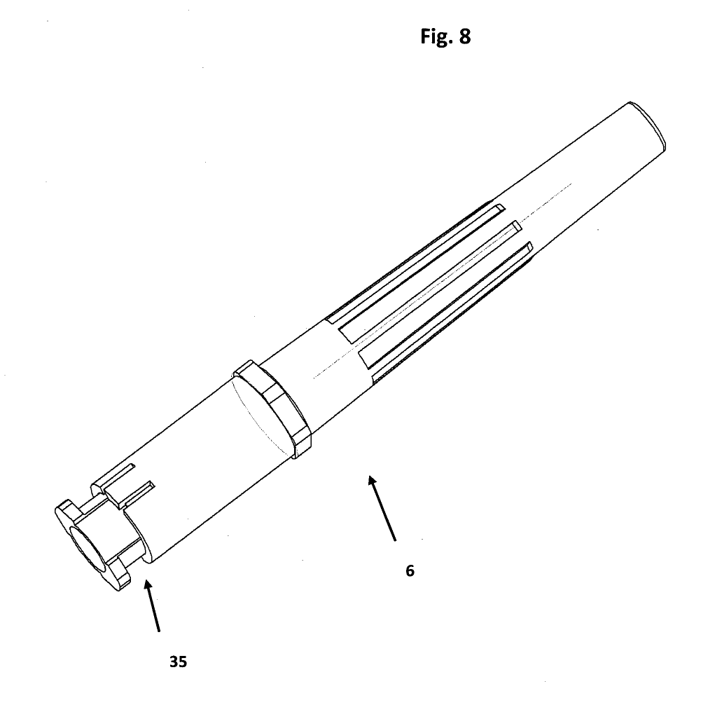

The draw-up needle 10, injection needle 3, and syringe 1 may incorporate engagement means. The preferred embodiment of the engagement means comprise of complementary protrusions and intrusions of the surfaces. Embodiments of the engagement of the draw-up needle 10, injection needle 3, and cap 6, are illustrated in FIGS. 5 to 14. A first embodiment is shown in FIGS. 5 to 6 and a second in FIGS. 7 to 14. Most conveniently the draw-up needle is easily unlocked by rotating the draw-up needle by 90 degrees in the preferred embodiments. The locking system preferably and most advantageously incorporates a quick release system which requires no more than one-quarter turn to engage or disengage the draw-up needle and injection needle members. This is much more convenient than the Luer-lock type engagement systems known in the art. By unlocking the draw-up-needle, the fluid bore 9 is automatically blocked in the embodiments shown in FIGS. 1-6. In these embodiments, the intrusions 16 of the draw-up needle engage with extrusions 13 of the needle hub and drive the needle hub simultaneously with the movement of the draw-up needle.

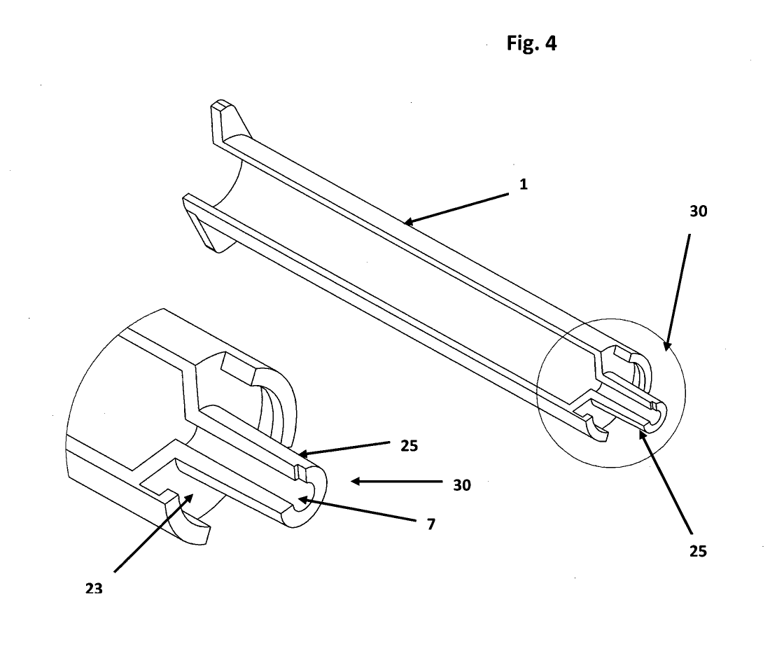

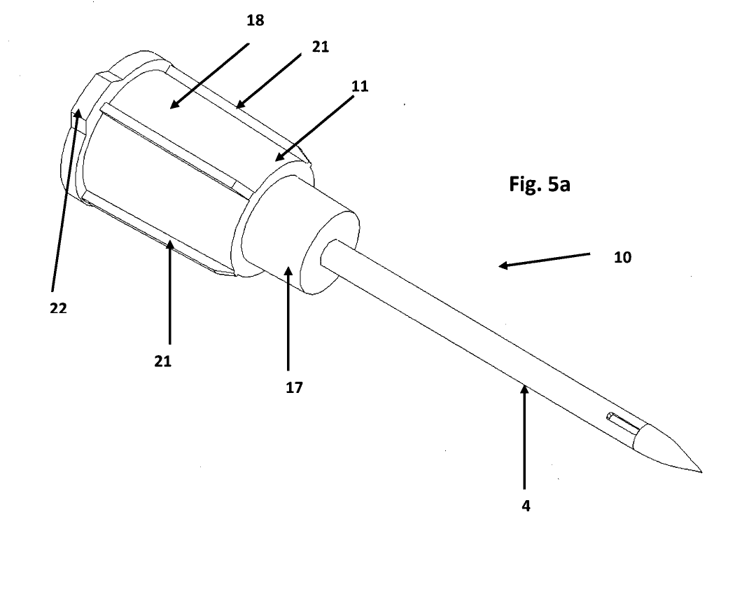

The engagement means may comprise of lugs 22 on the draw-up needle 10 as shown in FIGS. 5a and 5b and an undercut 23 at the distal end 30 of the syringe 1, shown in FIG. 4. The lugs 22 on the draw-up needle 10 form a broken ring which can be inserted into the undercut 23, which preferably defines a channel having an extension 24 to securely engage the lugs 22 of the draw-up needle.

The draw-up needle 10 incorporates a hub 11 which slidingly engages the injection needle hub 2. The injection needle hub 2 comprises of two portions, a distal portion 12 which has engagement protrusions 13 for engaging the draw-up needle hub 11 and a proximal portion 14 incorporating the bore 9. The internal surface 15 of the draw-up needle incorporates cut-outs or intrusions 16 defining a surface complementary to the exterior surface of the distal portion 12 of the injection needle hub 13. When the draw-up needle slides over the protrusions 13 of the hub 12 of the injection needle the complementary protrusions and intrusions engage to form an effective seal. Preferably the protrusions 13 are rib-shaped and the intrusions 16 complementary cut-out rib shapes to form an effective seal against fluid loss. The width of the ribs may be variable but the complementary surfaces on the injection needle hub and draw-up needle hub should be as close as possible for the best seal. Further, hub protrusions 13 and intrusions 16 should have a close fit to minimise the dead space.

The draw-up needle 10 engages with the reservoir or syringe barrel by way of an engagement means as shown in FIG. 4. Preferably the barrel of the syringe 1 is tapered at the distal end 25. Preferably the internal surface 31 of the proximal portion of the hub of draw-up needle defines a smooth complementary taper so that sliding engagement of the distal end 25 of the syringe barrel 1 with the smooth internal surface 15 of the draw-up needle 10 forms a leak-proof seal.

In one embodiment, the engagement means of the injection needle, draw-up needle, and syringe barrel co-operate to provide an effective locking means. In another embodiment, the injection needle, draw-up needle, and cap co-operate to provide an effective locking means. In one embodiment, the draw-up needle is engaged with or effectively locked onto the syringe barrel by rotating the lugs of the distal portion of the draw-up needle into an engaging position so that they fit underneath the holding lugs of the syringe barrel. The locking engagement advantageously ensures the draw-up needle 10 is not likely to disengage unintentionally by a user. Further, the locking engagement helps to prevent accidental needle-stick injuries. In another embodiment, the injection needle, draw-up needle, and cap are effectively locked together by sliding the draw-up needle protrusion along an axial channel in the hub of the injection needle and rotating the protusion to engage with engagement surfaces or faces in the hub of the injection needle. The so-engaged injection needle hub and draw-up needle hub are not easily disengaged, thus helping to prevent accidental needle-stick injuries. The frictional engagement of the cap with the hub of the draw-up needle in either embodiment makes it less likely that a needle-stick injury will occur from the draw-up needle.

FIG. 2 shows the elements in position for drawing fluid into the syringe 1 in one embodiment. When in position, the draw-up needle forms an effective safety cannula for the injection needle. The draw-up needle hub and injection needle hub 2 and tapered distal end 25 of the syringe engage concentrically, with the hub of the draw-up needle outermost, the hub of the injection needle in the middle, and the distal end of the syringe innermost. When engaged, the inner surface of the distal end 25 of the syringe and internal surface of the injection needle define a channel in concentric fluid communication with the channel defined by the draw-up needle and bore.

Preferably, the draw-up needle incorporates a side port 18 for transmission of fluid as shown in FIG. 5b. Preferably the injection needle incorporates a side port 19 for transmission of fluid as shown in FIG. 3. Other conformations are possible, but side ports advantageously eliminate the need for coring of needles during the manufacturing process. For example, the needle tip invention disclosed in WO 2008/074065, incorporated by reference herein in its entirety, may advantageously be incorporated into the injection needle.

It will be understood that the scope of the invention includes variable combinations of the elements described herein. For example, a cap may not be present as herebefore described, or the injection needle hub may not be present. Where there is no injection needle hub, the injection needle may be mounted directed on the reservoir. In one embodiment, the invention most advantageously includes an injection kit as shown in FIG. 6. Preferably the injection kit 30 comprises of a syringe 1 having a piston 5, a draw-up needle 10, an injection needle (not shown), and a container 31, wherein the syringe includes at least two channels in fluid communication. Preferably the container 31 is a blister package. Preferably the kit includes a cap 6, a draw-up needle 10, and an injection needle one within the other. The kit may include draw-up needle, injection needle and cap with engaging or locking mechanisms as herein described.

Any or all elements of the kit may be comprised of polymeric materials. In this embodiment, the elements of the kit are easily disposed of when used. Most advantageously, a kit comprising of polymeric materials may be collected and simply incinerated, for example, to minimise the potential for cross-contamination or unintentional transfer of pathogens acquired by any element of the kits during a reconstitution or injection process using the kit elements.

Another embodiment of the invention, shown in FIGS. 7 to 14 comprises of elements of a draw-up and injection system, including a draw-up needle 10, hypodermic or injection needle 3, and protective cap 6, for engaging with a syringe barrel such as that shown in FIG. 1. The elements of the draw-up and injection system are conveniently engageable as an effectively locked system, controlling the disengagement of the injection needle and the possibility of inadvertent contamination as described above. The system most advantageously enables the transfer of materials such as reconstitution of materials described above for injection without exposing the injection needle before it is needed for injection. Some embodiments of this may include the bore channel as described above.

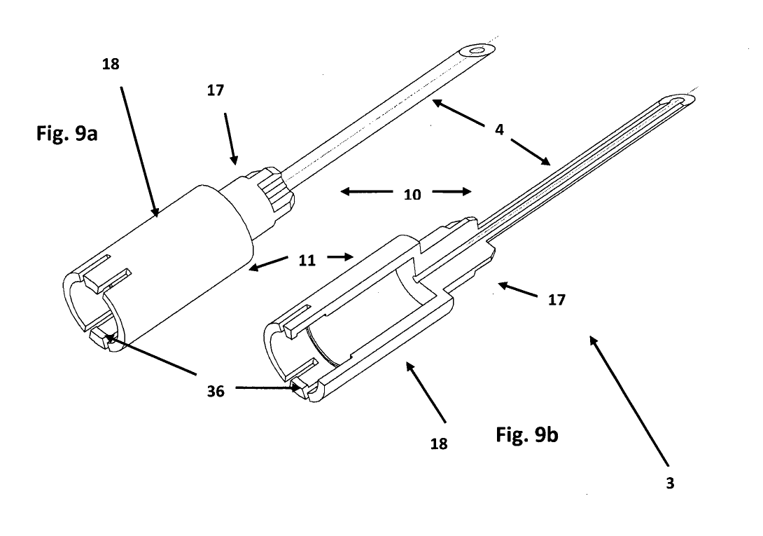

In one embodiment shown in FIGS. 7 and 8, the protective cap 6 engages securely with a frictional fit between the cap 6 and draw-up needle 10. FIG. 7 shows an exploded view of the injection needle 3 and injection needle hub 2, the injection needle hub incorporating a distal portion 35. The draw-up needle 10 may be securely engaged with both the injection needle 1 by engaging a securing or locking mechanism employing the hub of the injection needle 2 and the hub of the draw-up needle 11. FIG. 8 shows the elements in secure engaged position, the cap 6 and distal portion 35 of the injection needle hub 2 being visible.

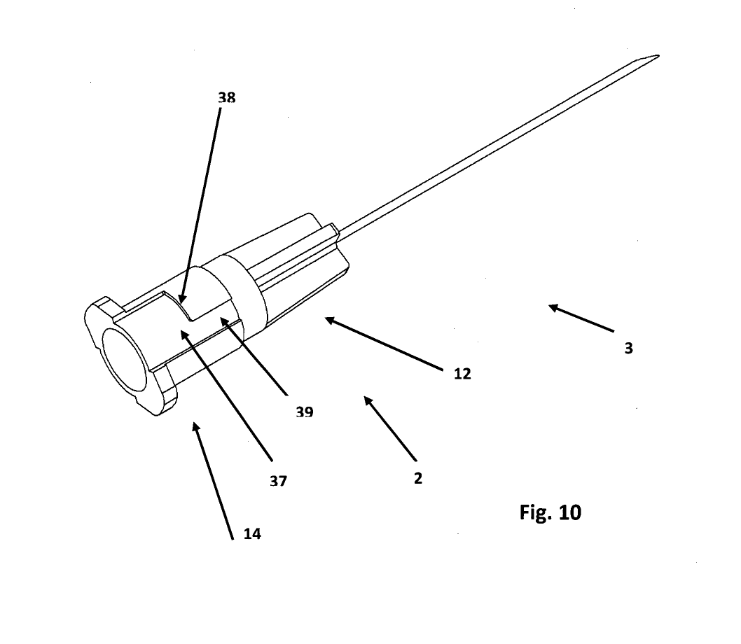

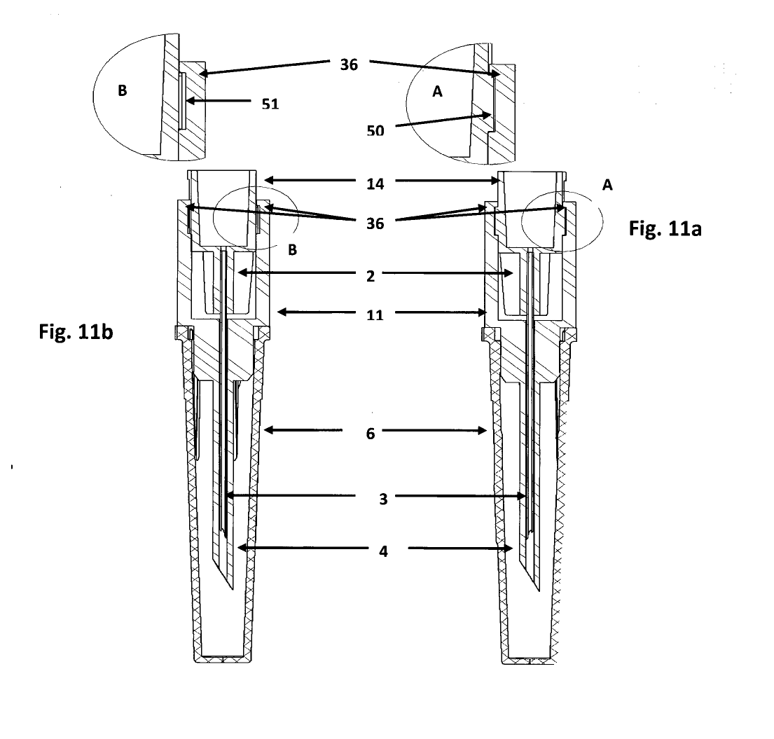

FIG. 9a shows an embodiment of a draw-up needle 10 in perspective view and FIG. 9b shows in perspective view the embodiment with a portion of the draw-up needle 10 cut away to illustrate the hub portions, including a distal portion 17, and a proximal portion 18 of the draw-up needle hub 11. The distal portion 17, includes a reduced diameter for receiving and engaging a protective cap 6. The internal surfaces of the cap 6 are formed to frictionally engage the external surfaces of the draw-up needle hub 11. The proximal portion 18 includes at least one undercut or protrusion 36 for engaging the hub of the injection needle.

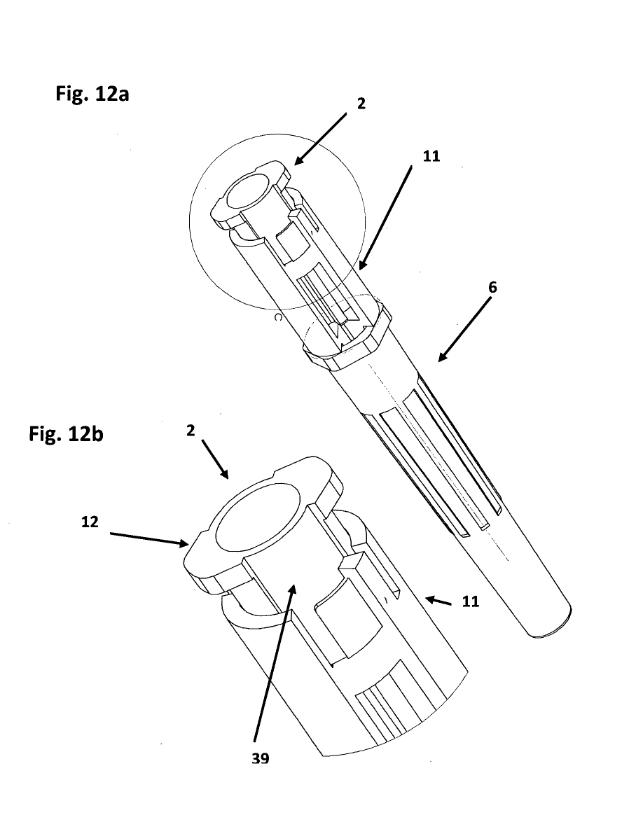

FIG. 10 shows in perspective view an embodiment of an injection needle 3 incorporating a hub 2 with engagement means for engaging the hub 11 of a draw-up needle, the injection needle hub 2 including a distal portion 12 and a proximal portion 14. Preferably, the engagement means of the injection needle hub 2 incorporates a channel 39 for directing a draw-up needle protrusion 36 with engagement means on the injection needle hub 2. Preferably, the engagement means of the injection needle hub 2 comprises of an engagement face 38 for engaging the draw-up needle protrusion 36. This embodiment most conveniently and advantageously allows a user to direct the draw-up needle protrusion 36 along the channel and twist the draw-up needle to engage the protrusion 36 with the engagement face 38 of the injection needle hub 2. When so engaged, the injection needle 3 and draw-up needle 10 form a unit.

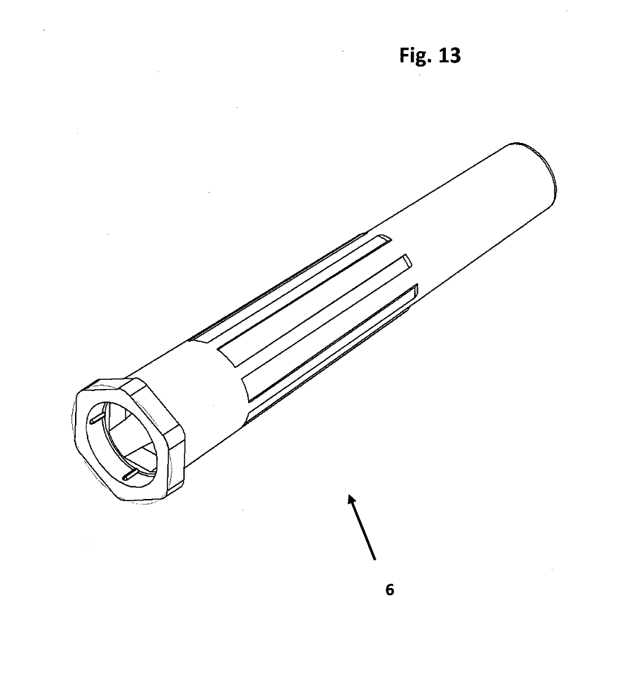

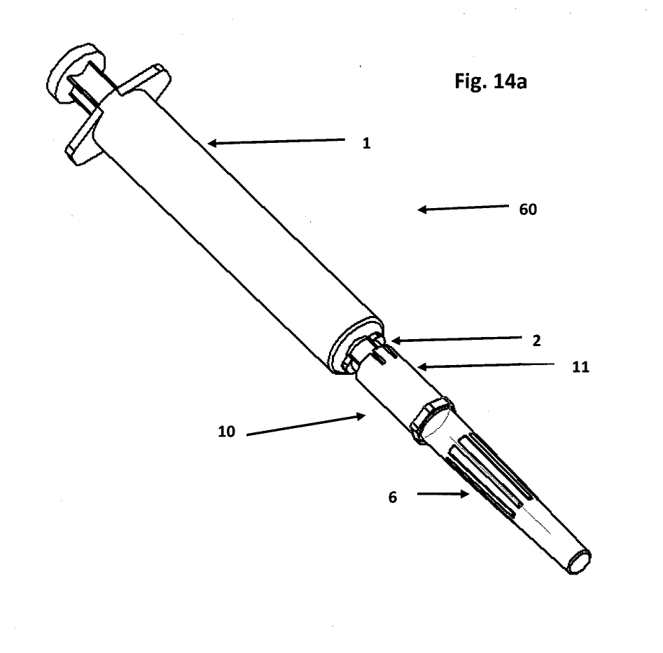

FIGS. 11a and 11b shows in transverse longitudinal section an embodiment of the engagement and disengagement positions of the draw-up needle hub 11 and the proximal portion 14 of the injection needle hub 2. In FIG. 11a, the draw-up needle hub 11 and injection needle hub are in engaged position, which is shown in larger detail in section A, where the protrusion 36 at the end of the draw-up needle hub 11 forming and is adjacent the complementarily-shaped or undercut surface 50 of the proximal portion 14 of injection needle hub. FIG. 11b shows a gap where the draw-up needle hub 11 has been rotated away from the engagement face 37 so that the draw-up needle hub 11 may be slideably disengaged along the channel from the injection needle hub.

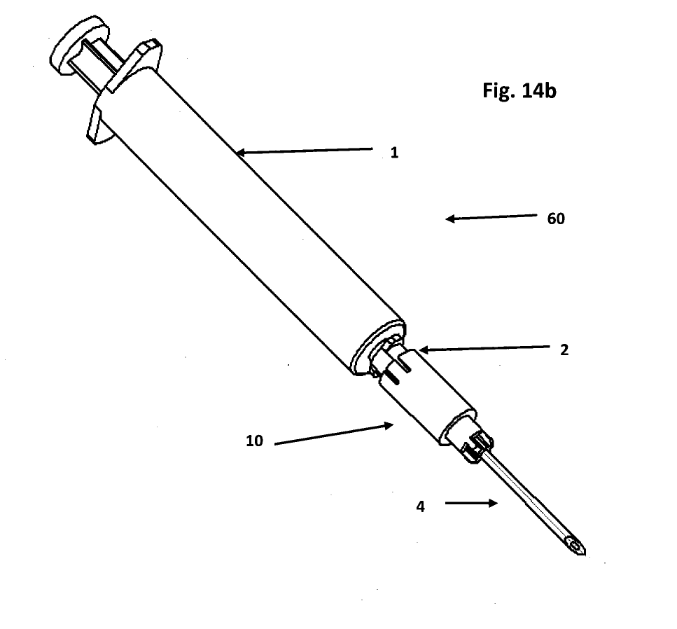

FIG. 12a shows further detail of an embodiment of the injection needle hub 2, draw-up needle hub 11 and cap 6 engaging. FIG. 12b shows in expanded view the draw-up needle hub 11 in a position moving axially along the channel 39 into position for engagement at the distal end 12 of the injection needle hub 2. (FIG. 12b is a scaled section view of FIG. 12a showing the engagement of the under-cut in perspective view)

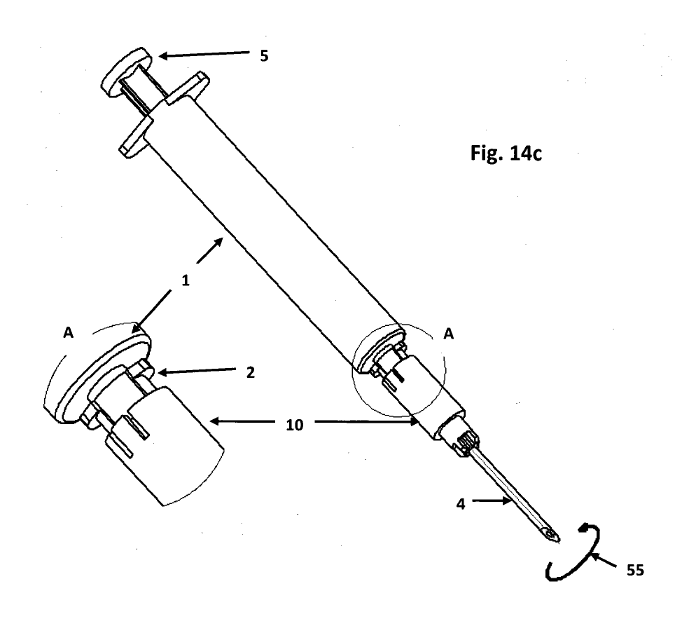

FIG. 13 shows a perspective view of a cap 6 which may be incorporated in the locking syringe system. The cap 6 may have ridges or protrusions on its surface to assist with disengaging the needles and their hubs for drawing up fluid into the barrel or reservoir of a syringe.

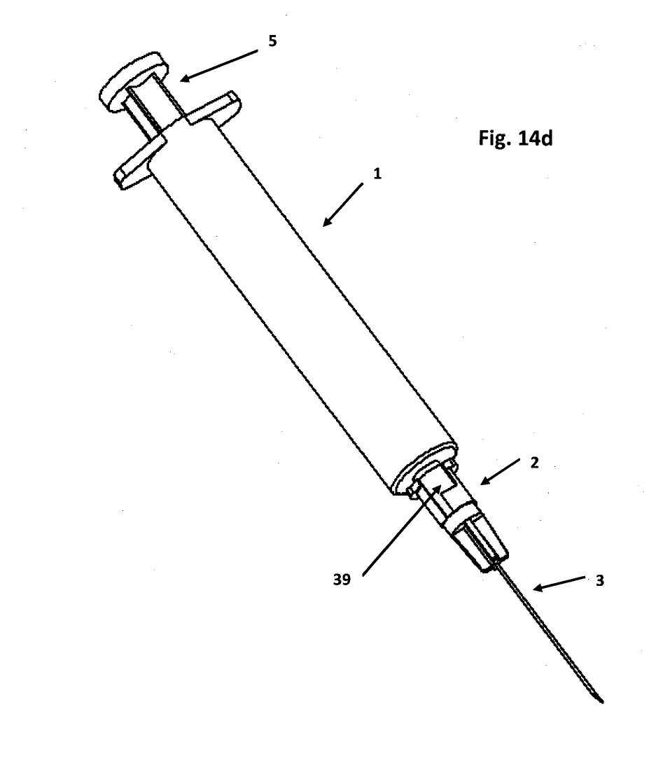

FIGS. 14a-14d illustrate the simple and easy steps of engaging the elements of an embodiment of a locking syringe system according to the invention and filling it with fluid for injection. FIG. 14a shows a locking syringe system 60 in perspective view, comprising of a syringe barrel 1, injection needle (hub 3 is visible), draw-up needle 10 (hub 11 is visible), and cap 6 with all members engaged. The embodiment including packaging as part of a kit as shown in FIG. 6. In the preferred embodiment, the injection needle hub 3 engages the syringe with a Luer lock or Luer slip. The syringe barrel 1 may have any suitable volume. In operation, the cap 6 is removed in a first step, if a cap is present as shown in the illustration. However, the kit may not include a cap and this step may not be necessary. Once the cap, if present, is removed, the syringe system 60 is ready for filling from a vial or reservoir containing fluid and materials in a next step to be transferred for mixing or for injection. This is shown in FIG. 14b where the draw-up needle cannula 4 is now visible. In the next step (FIG. 14c) the draw-up needle 4 is rotated 55 to a stopped position at a stop face 37. It will be understood that the rotation 55 direction will be dependent on the positioning of the channel and stop faces. The draw-up needle hub 10 can then be fully disengaged in a next step from the injection needle 3 by axial movement along the channel 39 on the hub 2 of the injection needle 3 (channel 39 shown in FIG. 10). The injection needle 3 and syringe 1 are now ready for use injecting as shown in FIG. 14d. It can be seen from the description hereabove that the method of invention provides steps of disengaging members using both rotational and translational or axial movement.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. Apparatus for injecting fluid comprising of:
an injection needle; and
a draw-up needle;
wherein said injection needle and said draw-up needle are reversibly engageable.

19. The apparatus of claim 18 further comprising a cap for engaging said draw-up needle.

20. The apparatus of claim 18 further comprising of a hub on said injection needle, said hub incorporating a channel for guiding engagement means on said draw-up needle.

21. The apparatus of claim 18 wherein said engagement means is a protrusion on said hub of said draw-up needle.

22. The apparatus of claim 20 further comprising an engagement face on said injection needle hub.

23. Apparatus according to claim 18 wherein at least one needle comprises of polymeric material.
